SEQUENCE LISTING (4 sequences)

SEQ ID NO: 1 - RNA, 34 nt, Artificial Sequence
  Description of Artificial Sequence Synthetic oligonucleotide
ggcgacccugaugaggccgaaaggccgaaaccgu

SEQ ID NO: 2 - RNA, 13 nt, Artificial Sequence
  Description of Artificial Sequence Synthetic oligonucleotide
acggucggucgcc

SEQ ID NO: 3 - DNA, 27 bp, Artificial Sequence
  Description of Artificial Sequence Synthetic oligonucleotide
ttttttttttttttttttttttttttt

SEQ ID NO: 4 - RNA, 13 nt, Artificial Sequence
  Description of Artificial Sequence Synthetic oligonucleotide
acggucggucgcc

(19) United States
(12) Patent Application Publication
(10) Pub. No.: US 2006/0211018 A1
(43) Pub. Date: Sep. 21, 2006

(54) Nucleozymes and methods of use

(75) Inventors: Benjamin G. Schroeder, San Mateo, CA (US); Stefan M. Matysiak, El Granada, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 11/350,304

(22) Filed: Feb. 8, 2006

Related U.S. Application Data
(60) Provisional application No. 60/651,158, filed on Feb. 8, 2005.

(51) Int. Cl. C12Q 1/68; C07H 21/04

(52) U.S. Cl. 435/6; 536/25.32

(57) ABSTRACT

Disclosed herein are nucleozymes and methods of use in the detection and analysis of target polynucleotide sequences.

### 1. CROSS REFERENCE TO RELATED APPLICATION

This application claims benefit under 35 U.S.C. § 119(e) to application Ser. No. 60/651,158, filed Feb. 8, 2005, the contents of which are incorporated herein by reference.

### 2. BACKGROUND

The sensitive detection and quantitation of specific nucleic acid sequences has widespread application and is becoming increasingly important in basic research and diagnostics and genomics. The methods of nucleic acid detection commonly employed are limited by the number of nucleic acid sequences that can be simultaneously assayed. This is a result of the limited number of catalytic proteins and detection molecules than can be employed in these assays. Therefore, there is a need in the art for methods of nucleic acid sequence detection and quantitation that provide greater flexibility and broader application in the number of and types of nucleic acid sequences that can be detected.

### 3. SUMMARY

The present disclosure provides methods and compositions for detecting and quantitating target polynucleotides. In general, the methods are designed to produce a polynucleotide comprising a nucleozyme moiety if a target polynucleotide is present in a sample. The nucleozyme moiety is enzymatically active. Therefore, detection or quantitation of the enzymatically active nucleozyme moiety can be used as an indicator of the presence and quantity of a target polynucleotide in a sample.

A polynucleotide comprising a nucleozyme moiety can be produced by various techniques. In some embodiments, a target polynucleotide can be amplified using forward and reverse amplification primers to produce a double stranded DNA amplicon. One of the primers can comprise a sequence complementary to a nucleozyme moiety. Therefore, during amplification a nucleozyme moiety can be incorporated into the opposite strand of the amplicon. In embodiments, wherein the nucleozyme moiety is catalytically active as DNA (deoxyribonucleozyme), the two strands of the double stranded amplicon can be separated and the enzymatic activity of the nucleozyme moiety can be assayed.

In some embodiments, a target polynucleotide can be amplified using forward and reverse amplification primers to produce a double stranded DNA amplicon. One of the primers can comprise a sequence complementary to a ribonucleozyme moiety and the other primer can comprise an RNA promoter sequence. Therefore, as a result of amplification a double stranded template suitable for RNA transcription can be produced. Therefore, transcribing the double stranded template with a suitable RNA polymerase yields a polynucleotide comprising a ribonucleozyme moiety.

The catalytic activity of the nucleozyme moieties can be assayed by various techniques. In some embodiments, the nucleozyme moieties can be assayed by cleavage of an oligonucleotide substrate. In some embodiments, an oligonucleotide substrate can comprise a reporter system suitable for producing a detectable signal. In various exemplary embodiments, a signal can be a fluorescent or chemiluminescent signal. The reporter system can be designed to be activated when the oligonucleotide substrate is cleaved by a nucleozyme moiety. In some embodiments, an oligonucleotide substrate can comprise a hydrophobic moiety that forms a precipitate when the oligonucleotide substrate is cleaved. In some embodiments, a label suitable for producing a detectable signal co-precipitates with the hydrophobic moiety.

In some embodiments, nucleozyme moieties can be assayed in singleplex or multiplex formats. In some embodiments, nucleozyme moieties can be assayed in solution or hybridized to a capture probe attached to a surface. In some embodiments, a surface can comprise a plurality of capture probes each hybridized to a different polynucleotide comprising a nucleozyme moiety.

### 4. BRIEF DESCRIPTION OF THE FIGURES

The figures described below, are for illustration purposes only and are not intended to limit the scope of the present disclosure in any way;

FIG. 1 provides a drawing illustrating an embodiment of a ribozyme comprising Stems I-III and U-turn bound to an oligonucleotide substrate;

FIG. 2 provides a cartoon illustrating an embodiment of making a template from which sequence tagged ribozymes are transcribed;

FIG. 3 provides a cartoon illustrating an embodiment in which a ribozyme comprising a sequence tag is hybridized to a capture probe array and catalyzes the cleavage of an RNA oligonucleotide substrate comprising a fluorescer/quencher pair. A fluorescent signal is produced upon cleavage of the substrate;

FIG. 4 provides an illustration of an embodiment in which dioxetane is released from an oligonucleotide substrate following cleavage by a ribozyme and an intramolecular reaction to produce a chemiluminescent signal;

FIG. 5 provides an illustration of an embodiment in which an dioxetane labeled ribonucleotide can be synthesized;

FIG. 6 provides a cartoon illustrating of an embodiment of making a sequence tagged DNA enzyme;

FIG. 7 provides a cartoon illustrating an embodiment of making a template from which sequence tagged ribozymes are transcribed; and

FIG. 8 provides the structure of an exemplary phosphotriester.

### 5. DETAILED DESCRIPTION

In this application, the use of the singular includes the plural unless specifically stated otherwise. The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Disclosed herein are methods and compositions for detecting one or more target polynucleotides in a sample. In general, the disclosed methods and compositions are designed to produce polynucleotides derived from target polynucleotides that can be single stranded or double stranded and engineered to include one or more features suitable for their manipulation and detection. The methods are designed such that the synthesis of a “derivative polynucleotide” depends upon the presence of a target polynucleotide in a sample. In some embodiments, a derivative polynucleotide can comprise a “nucleozyme moiety” (e.g., a nucleic acid enzyme sequence, catalytic enzyme sequence). In some embodiments, the disclosed methods and compositions can be designed to produce a derivative polynucleotide comprising a nucleozyme moiety and a target polynucleotide sequence or a sequence complementary thereto. The nucleozyme moiety comprises catalytic activity for carrying out various reactions suitable for detection. The target polynucleotide sequences or target polynucleotide complementary sequences of the derivative polynucleotides can be used as “sequence tags” for downstream processing, including but not limited to, isolation and detection.

Thus, the disclosed methods and compositions can be used to produce a nucleic acid enzyme comprising a “sequence tag” when a target polynucleotide is present in a sample. Therefore, detection of a “tagged” catalytic nucleic acid can be indicative of the presence of a target polynucleotide. In various embodiments, the sequence tag can be attached to the 5′ or 3′ terminus of a nucleic acid enzyme and can comprise a target polynucleotide sequence or a sequence complementary to a target polynucleotide. In some embodiments, the sequence tag can be used to distinguish the disclosed nucleic acid enzymes from other catalytic nucleic acids that do not comprise sequence tags.

“Nucleic acid enzyme”, “catalytic nucleic acid”, “nucleozyme”, “nucleozyme moiety” as used herein refer to nucleobase polymers that catalyze a chemical reaction and therefore include but are not limited to catalytic DNA, RNA, and mixed and synthetic nucleobase polymers. Tagged nucleic acid enzymes produced by the disclosed methods can be detected by various methods as known in the art. In some embodiments, a tagged nucleic acid enzyme can be detected by assaying for its catalytic activity. In some embodiments, the catalyzed reaction can occur in solution. In some embodiments, the reaction can occur while the tagged nucleic acid enzyme is hybridized to a capture probe that optionally can be attached to a surface. In some embodiments, the sequence tag can be used to hybridize the nucleic acid enzyme to a capture probe. In some embodiments, the surface to which a capture probe can be attached comprises an array of capture probes.

The reactions catalyzed by tagged nucleic acid enzymes that can be suitable for detection of a tagged nucleic acid enzyme are known in the art. In general, these reactions comprise modification(s) of one or more oligonucleotide substrates. The modifications can include but are not limited to endonuclease cleavage, ligation, biotinylation, acylation, sulfur alkylation, phosphorylation, dephosphorylation, methylation, demethylation, and polymerization. (see, e.g., Landweber et al. “Ribozyme Engineering and Early Evolution” BioScience 48(2) (1989)) Therefore, the detection of a reaction a product of a tagged nucleic acid enzyme can be used to correlate with the presence of a target polynucleotide.

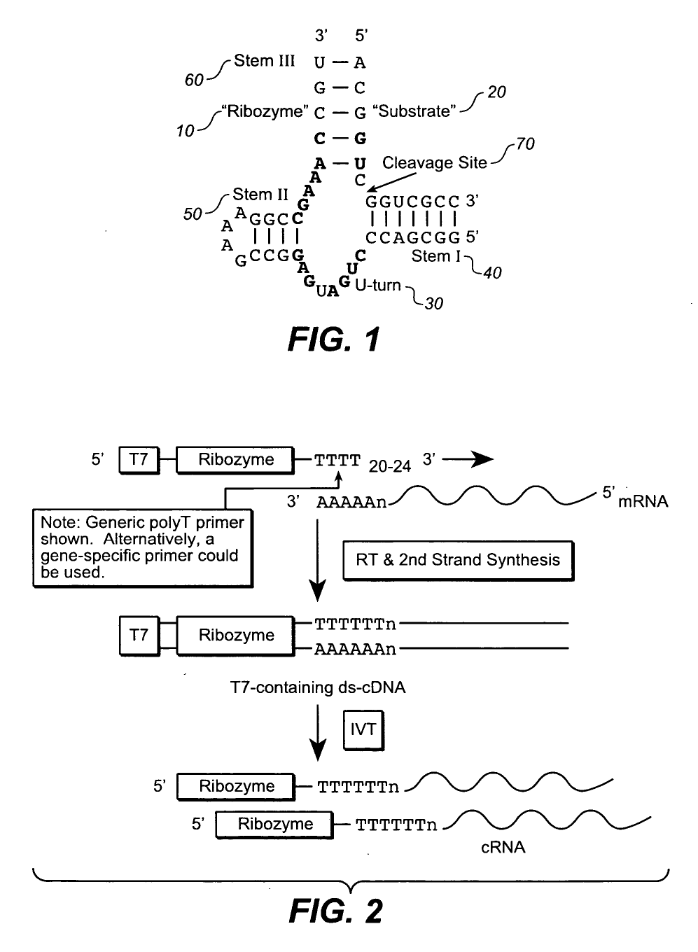

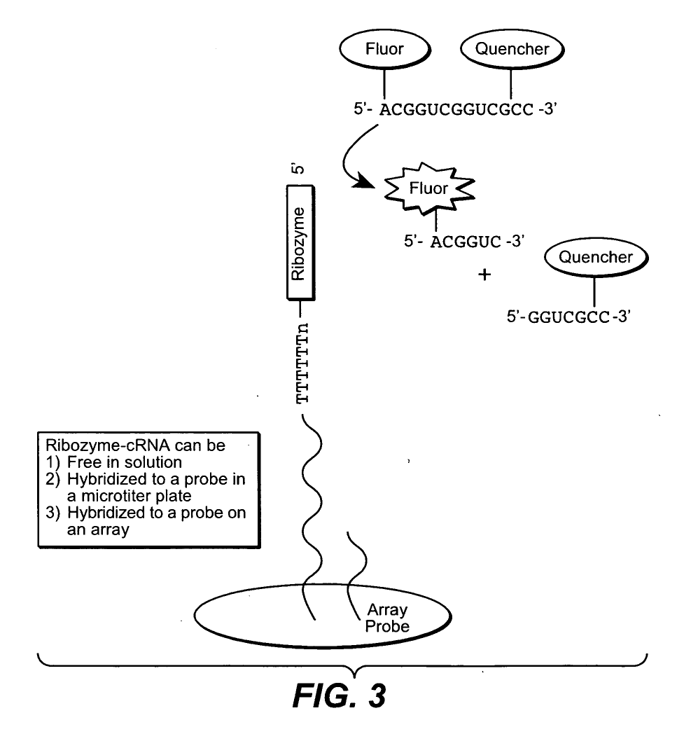

For example, in some embodiments nucleic acid enzyme can comprise hammerhead ribozyme 10. When hybridized to oligonucleotide substrate 20 the ribozyme sequence comprises U-turn 30, and Stem 140, Stem II 50, and Stem III 60 (FIG. 1). Stems I and III hybridize to oligonucleotide substrate 20 which is cleaved at cleavage site 70 by ribozyme 10. The substrate oligonucleotide cleavage products and ribozyme disassociate.

In some embodiments, a modified oligonucleotide substrate can be detected using a reporter molecule suitable for producing a detectable signal (e.g., a fluorescent signal or chemiluminescent signal). Therefore, in some embodiments, an oligonucleotide substrate can comprise one or more labels that can be used to detect a reaction product (i.e., a modified substrate). In some embodiments, a label can comprise a reporter molecule that can be activated when an oligonucleotide substrate is modified by the tagged nucleic acid enzyme. Therefore, in some embodiments, a reporter molecule can be suitable for monitoring the enzymatic activity of a tagged nucleic acid enzyme in real-time. In various exemplary embodiments, a reaction catalyzed by a tagged nucleic acid enzyme can attach or remove a reporter molecule from an oligonucleotide substrate (e.g., ³²P). Therefore, in various exemplary embodiments, an increase or decrease in a detectable signal during the course of a reaction catalyzed by a tagged nucleic acid enzyme can be used in the disclosed methods.

In some embodiments, other types of molecules can be attached to an oligonucleotide substrate. For example, molecules that modulate the hydrophobicity or solubility of a reaction product can be used. For example, in some embodiments, a hydrophobic moiety can be attached to an oligonucleotide substrate. In some embodiments, the hydrophobic moiety does not substantially reduce the solubility of an oligonucleotide substrate but upon cleavage of the substrate the cleavage product having the hydrophobic moiety can form a precipitate. In some embodiments, the precipitated cleavage product also can comprise a label to facilitate detection and analysis. Therefore, the precipitate can be used to localize a detectable signal when assaying for the enzymatic activity of a tagged nucleic acid enzyme that is hybridized to a capture probe. Although the use of labels, reporter molecules, and other types of moieties that facilitate detection and analysis can be used in the disclosed methods, the skilled artisan will appreciate that in some embodiments, a modified oligonucleotide substrate can be detected directly without the use of such detection aids (e.g., capillary electrophoresis).

The methods described herein can be carried out in various modes or formats. For example, in some embodiments, a single target polynucleotide of interest can be detected in a “single-plex” mode. The methods described herein also can be carried out in a “multiplex” mode, in which a plurality of different target polynucleotides can be simultaneously detected. In some multiplex embodiments, each nucleic acid enzyme can catalyze a reaction that can produce signals that can be distinguished (e.g., spectrally resolvable signals). In some embodiments, multiplexing can be achieved by producing a plurality of tagged nucleic acid enzymes that each catalyze a different reaction and/or that each react with a substantially unique substrate. In some embodiments, a plurality of tagged nucleic acid enzymes can be produced in a single multiplex reaction and aliquots from the reaction can be transferred to individual wells of a multiwell plate, each comprising a single substrate. Therefore, the wells can be scanned and individual wells providing a positive reaction can be used as an indicator of the presence of a target sequence in a sample. Determining the number of target polynucleotides that can be detected in a multiplex format is within the abilities of the skilled artisan and is dependent at least in part on the number and types of target sequences to be detected, the types of nucleic acid enzymes employed and the reactions they catalyze, and the methods that can be used to distinguished the reaction products of the tagged nucleic acid enzymes.

The methods disclosed herein in which the synthesis of a tagged nucleic acid enzyme depends on the presence of a target polynucleotide in a sample can be accomplished using various methods and techniques. In some embodiments, a tagged nucleic acid enzyme can be produced by hybridizing a primer comprising an enzymatically active nucleic acid sequence to a target polynucleotide and extending the primer using a polymerase. Therefore, in some embodiments, an extended primer can comprise a tagged nucleic acid enzyme. In various exemplary embodiments, the enzymatically active nucleic acid sequence can be any nucleobase polymer having enzymatic activity and, therefore, can be DNA, RNA, a mixed polymer, and/or can comprise non-naturally occurring nucleotide moieties, as described below. The sequence added to the primer by the polymerase comprises a “sequence tag” which in this exemplary embodiment is complementary to the target polynucleotide. The skilled artisan will appreciate that in this and other non-limiting embodiment, described below, a primer can be enzymatically active prior to annealing and extension. In some embodiments, the catalytic activity of the primer can be substantially inhibited by carrying out the annealing and extension under conditions that are unsuitable for catalysis (e.g, at about 68° C.). To distinguish the enzymatic activity of a tagged nucleic acid enzyme from an unextended primer, the tagged nucleic acid enzyme can be isolated or purified. Methods of isolated nucleic acids include, but are not limited to, chromatography, electrophoresis, and the like, and can be used to enrich, purify, or isolate the tagged enzyme sequence. In some embodiments, this can be accomplished using a capture probe that is substantially complementary to the sequence tag, as further described below.

“Primer” as used herein refers to an oligonucleotide or polynucleotide capable of hybridizing or annealing to a template polynucleotide to form a substrate for a polymerase (e.g., DNA-dependent DNA polymerases, RNA-dependent DNA polymerase (reverse transcriptases), thermostable polymerases (e.g., Taq polymerase)). Therefore, in various embodiments, a primer can include but is not limited to an amplification primer (e.g., forward and reverse primers) and/or a reverse transcription primer and/or a complementary strand primer, etc. “Annealing” or “hybridizing” refer to base-pairing interactions of one nucleobase polymer with another that results in the formation of a double-stranded structure. In some embodiments, annealing occurs via Watson-Crick base-pairing interactions, but may be mediated by other hydrogen-bonding interactions, such as Hoogsteen base pairing. (see S. O. Doronina & J.-P. Behr Chemical Society Rev. 1997, 63 ff; Brotschi et al., 2001, Angew. Chem. Int. Ed. 40(16):3012; Kool, 2001, Ann. Rev. Biophys. Biomol. Struct. 30:1-22; Hikishima et al., 2005, Angew. Chem. Int. Ed. 44:596-598; Hill et al. 2001, Chem. Rev. 101:3893-4001) When a primer is hybridized to its template (e.g., a target polynucleotide) in the presence of deoxynucleotide triphosphates (dNTPs), a polymerase can initiate synthesis of a nascent polynucleotide strand in a template directed manner beginning at the 3′-OH terminus of the primer, as known in the art. In some embodiments, a primer can comprise non-naturally occurring nucleotides or nucleobases as described herein.

Therefore, in some embodiments, a primer can be a “sequence specific primer”. By “sequence specific primer” as used herein refers to a primer that does not generally anneal or hybridize to multiple target polynucleotide sequences. However, the skilled artisan will appreciate that in some embodiments a sequence of a sequence specific primer can be shared by a plurality of primers. In some embodiments, a primer can be a degenerate primer and can hybridize to multiple target polynucleotide sequences (e.g., poly-T FIG. 2). Therefore, in various exemplary embodiments a primer can comprise a domain or sequence suitable for hybridizing to one or more target polynucleotides. Once hybridized, a primer can be extended by a polymerase to produce a nascent sequence that is complementary to the target polynucleotide. In some embodiments, primers can be forward or reverse primer pairs. “Forward primer” as used herein refers to a primer comprising a sequence complementary to a target sequence. Thus, a forward primer can hybridize to a target sequence and can be extended by the action of a polymerase to produce a “forward strand”. “Reverse primer” as used herein refers to a primer comprising a sequence that can hybridize to the extended forward primer. Thus, a reverse primer can hybridize to the region added to the forward primer by the polymerase. Extension of the hybridized reverse primer by a polymerase produces a “reverse strand”.

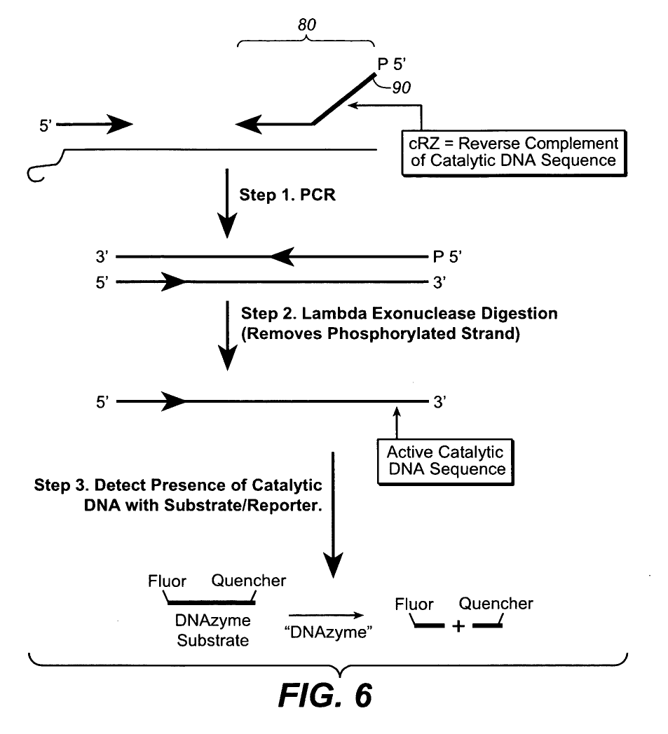

In addition to a target specific sequence, the primers disclosed herein can comprise a plurality of domains or sequences suitable for specific purpose. For example, in some embodiments, a primer can comprise a nucleic acid enzyme sequence. In some embodiments, a primer can comprise a sequence complementary to a nucleic acid enzyme sequence. As shown in FIG. 6, primer 80 comprises cRZ 90, reverse complement of a catalytic DNA sequence. By “nucleic acid enzyme sequence” and “nucleozyme sequence” is meant a sequence that is the same polarity or complementarity of a catalytically active nucleic acid. Therefore, in some embodiments, a nucleic acid enzyme sequence can be enzymatically active. However, in some embodiments a nucleic acid enzyme sequence may not be substantially active. The skilled artisan will appreciate that a nucleic acid sequence having the same polarity as a catalytically active nucleic acid can be essential but alone may not be sufficient for catalytic activity. This is because in many instances the presence or absence of a 2′-OH in the ribose moiety of a nucleic acid can be an important condition of catalytic activity. Therefore, many sequences, for example, can be catalytic only as RNA or DNA and are not substantially catalytic when converted to the other form.

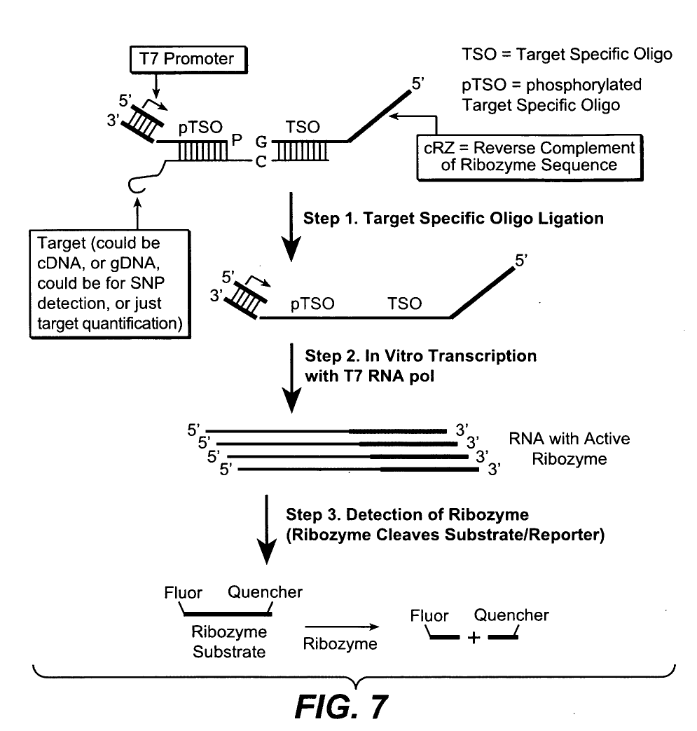

Therefore, in some embodiments a DNA primer can comprise a nucleic acid enzyme sequence that is converted to RNA to be catalytically active (see, e.g., FIG. 7). In some embodiments, this can be accomplished by synthesizing a template from which RNA transcripts comprising a tagged nucleic acid enzyme can be produced. To be suitable for this purpose, the template can be a double-stranded DNA that comprises a promoter suitable for initiating transcription by a DNA-dependent RNA polymerase (e.g., a T3, T7, and/or SP6 promoter). The promoter can be operably linked and positioned 5′ relative to the sequences that will comprise a tagged nucleic acid enzyme. In some embodiments (e.g., FIG. 2), the template can be produced by annealing a primer comprising a 5′ promoter sequence, a nucleic acid enzyme sequence, and 3′ target complementary sequence to a target polynucleotide. The primer can be extended by the action of a polymerase to produce a first template strand. A second primer can be annealed to the first strand and extended to produce the complementary strand and thereby complete the synthesis of the transcription template. Therefore, a tagged ribonucleic acid enzyme can be produced by transcribing the double-stranded template in the presence of a DNA-dependent RNA polymerase (e.g., T4 polymerase, T3 polymerase, T7 polymerase, and/or SP6 polymerase) and ribonucleotide triphosphates (rNTPs) suitable for RNA synthesis. The skilled artisan will appreciate that once initiated multiple rounds of transcription can ensue and therefore a tagged ribonucleic acid enzyme can be linearly amplified.

In some embodiments, a primer can comprise a sequence that is the reverse complement of a catalytically active nucleic acid enzyme sequence. Therefore, in some embodiments, a primer can be referred to as comprising a domain that “encodes” a nucleic acid enzyme. The skilled artisan will appreciate that a tagged nucleic acid enzyme can be produced from an encoded sequence by synthesizing that sequence's complementary strand. The complementary strand comprising the nucleic acid enzyme can be DNA or RNA depending on the type of nucleic acid enzyme that is encoded. Therefore, in some embodiments an encoded nucleic acid enzyme can be produced as an RNA transcript (i.e., cRNA) or a complementary DNA strand.

For example, in some embodiments, a primer comprising a target specific sequence and a domain that encodes for a nucleic acid enzyme can be annealed to a target polynucleotide and extended to produce a first strand. If the primer encodes for a DNA enzyme, the tagged enzyme can be produced by annealing and extending a primer complementary to the first strand. However, the skilled artisan will appreciate that a tagged nucleic acid enzyme is substantially inactive when hybridized to its complementary strand. Therefore, in some embodiments a tagged nucleic acid enzyme can be released from its complementary strand by various methods known in the art that disrupt the hydrogen bonding between two nucleobase polymer strands, such as, altering the temperature, pH, and/or ionic strength. In some embodiments, the strand hybridized to the tagged nucleic acid enzyme can be selectively inactivated and/or degraded. For example, in some embodiments, a primer comprising a domain that encodes for a nucleic acid enzyme can comprise a moiety that selectively renders it susceptible to an exonuclease. For example, in some embodiments, the moiety can comprise a 5′-PO₄ which renders a polynucleotide susceptible to digestion by 5′ exonucleases, such as, λ phage exonuclease. In some embodiments, a tagged nucleic acid enzyme and/or its complementary strand can comprise one or a plurality of moieties or labels suitable for selectively isolating one or both strands. For example, in some embodiments, a primer can comprise a magnetic label, including but not limited to a magnetic bead (see, e.g., Cotton et al., 1985, J. Am. Chem. Soc. 107:7438-7445; Agaskar et al. 1987, Inorg. Chem. 26:4051-4057; Cotton et al., 1991, Inorg. Chem. 30:2509-2514; Cotton et al., 1992, Inorg. Chem. 31:5308-5315) and therefore one strand can be selectively isolated by denaturing the double stranded molecule in a magnetic field.

In embodiments wherein a primer comprises a domain that encodes for an RNA enzyme, a tagged RNA enzyme can be produced as an RNA transcript from a DNA template. The tagged RNA enzyme can be transcribed from a double-stranded DNA template, as described above; however, a double-stranded DNA template is not required for transcription. For example, in some embodiments, a template suitable for transcription can be partially single-stranded and partially double-stranded. In some embodiments, a partially single- and double-stranded template can be produced using two or more probes that can be ligated (“ligation probes”) when annealed or hybridized to a target polynucleotide. Thus, “ligation probes” as used herein refers to polynucleotides capable of hybridizing or annealing to a template polynucleotide and are suitable to form a substrate for a ligase. Therefore, in some embodiments, a ligation probe can have a 3′-OH and/or a 5′-PO₄. Similar to primers, as described above, probes also can comprise a plurality of domains or sequences in addition to a target specific sequence. The probes can be ligated under isothermal conditions (e.g., T4 DNA ligase) of by thermocycling (thermostable ligase).

In some embodiments, probes can be used to produce a template suitable for transcription by annealing first and second ligation probes to a target polynucleotide. In some embodiments a first ligation probe comprises a 5′ target specific sequence and a 5′-PO₄. In some embodiments, the 3′ sequence of the first ligation probe can comprise a sequence complementary to a promoter. The skilled artisan will appreciate that a double stranded promoter is suitable to initiate transcription; however, the region that is transcribed can be single-stranded. Therefore, in some embodiments, a double-stranded promoter can be formed by annealing the complementary oligonucleotide to the 3′ promoter sequence. In some embodiments, a double-stranded promoter can be formed by a stem-loop structure at the 3′ terminus of the first probe. The stem-loop structure can be formed because the 3′ terminus comprises a promoter sequence and its complement. In some embodiments, a second ligation probe comprising a 3′ target specific sequence, a 3′-OH, and a 5′ sequence encoding for a ribonucleic acid enzyme can be hybridized to the target polynucleotide 5′ relative and immediately adjacent to the first probe without an intervening “gap”. Therefore, the two probes hybridize to the target polynucleotide such that the 5′-PO₄ of the first probe and 3′-OH of the second probe form a substrate suitable for ligation by a DNA ligase (e.g., T4 DNA ligase, thermostable ligase). Following ligation, the ligated probes and the double-stranded promoter form a template suitable for transcription of a tagged ribonucleic acid enzyme. If ligation does not occur, an RNA comprising a tagged nucleic acid enzyme is not produced.

In some embodiments, ligation probes can be separated by a gap of one or more nucleotides. In some embodiments, a gap between the hybridized probes may be filled-in by hybridizing one or more additional ligation probes comprising target specific sequences. In some embodiments, the gap can be filled-in by extending the 3′-terminus of a ligation probe hybridized to the target polynucleotide at a position 5′ relative to another ligation probe. In some embodiments, a specific dNTP(s) can be used to fill-in a gap. Therefore, detection of a tagged ribonucleic acid enzyme can be indicative of a specific nucleotide (e.g., single-nucleotide polymorphism (SNP)) or sequence in the gap region.

Based on the foregoing description of several exemplary embodiments, permutations of these methods for producing a tagged nucleic acid enzyme will be apparent to the skilled artisan. For example, in some embodiments, a tagged nucleic acid enzyme can be produced in a reverse transcription reaction.

In some embodiments, a tagged nucleic acid enzyme can be produced by hybridizing a target polynucleotide to a first ligation probe comprising a catalytically active nucleic acid enzyme sequence and a second ligation probe. Therefore, a tagged nucleic acid enzyme can be produced by ligating the two probes and the use of a polymerase is not required. Therefore, one or both ligation probes can comprise nucleotides that are unsuitable for use with a polymerase.

In some embodiments, a tagged nucleic acid enzyme can be produced by methods that employ the principles and techniques of PCR. For example, in some embodiments, a double-stranded DNA template suitable for transcription can be synthesized by PCR. Similarly, a double-stranded DNA in which one of the strands comprises a tagged nucleic acid enzyme can be exponentially amplified using forward and reverse primers. In some embodiments, asymmetric PCR can be used to produce excess copies of the strand that comprises the tagged nucleic acid enzyme. For example, a forward primer that hybridizes to the complement of the strand comprising the tagged nucleic acid enzyme can be in molar excess relative to the reverse primer. Therefore, amplification initially can be exponential when the forward and reverse primes are present. Following consumption of the reverse primer, linear amplification from the forward primer produces the strand comprising the tagged nucleic acid at a rate proportional to the number of double-stranded amplicons produced during the exponential amplification. (see, e.g., U.S. Patent Application No. 20030207266)

A reaction catalyzed by a nucleic acid enzyme may proceed to a steady state or may proceed to completion substantially faster than in the absence of the nucleic acid enzyme. In some embodiments, the nucleic acid enzyme can be substantially unaltered by the reaction and can therefore continue catalysis of virtually any number of reactions. In some embodiments, the rate and extent of catalysis can be limited through the use of inhibitors that substantially reduce catalysis by interfering with substrate/enzyme interactions or modify the nucleic acid enzyme as known in the art (e.g., formamide, urea and other denaturants, modification of reaction conditions (e.g., temperature), chelators (e.g., EDTA), enzymes and the like).

In general, nucleic acid enzymes are capable of hybridizing to one or more substrates, for example, by Watson-Crick base pairing and enzymatically modifying the substrate. Reactions catalyzed by nucleic acid enzymes include but are not limited to endonuclease cleavage of phosphodiester linkages (e.g., 3′,5′ and 2′,5′-phosphodiester linkages (Guerrier-Takada et al., 1983, Cell. 35(3 Pt 2):849-57; Ordoukhanian et al., 2002, J. Am. Chem. Soc. 124:12499-506; Shih et al., 1999, RNA. 5:1140-8)), ligation of phosphodiester linkages (e.g., 3′,5′ and 2′,5′-phosphodiester linkage (Bartel et al., 1993, Science 261(5127):1411-8; Bergman et al., 2000, Biochemistry 2000 Mar. 21; 39(11):3115-23; Ekland et al., 1995, Science 269(5222):364-70; McGuinness et al., 2002, Chem. Biol. 9:297-307; Vlassov et al., 2004, Nucleic Acids Res. 32(9):2966-74)), biotinylation, digoxigenin addition, acylation, sulfur alkylation, phosphorylation, dephosphorylation, methylation, or polymerization. Once modified, the substrate can be released from the nucleic acid enzyme which in some embodiments can catalyze additional reactions. In some embodiments, substrate binding and enzymatic activity can substantially reside in various domains, regions, or sequences of a nucleic acid enzyme. Therefore, in some embodiments, a nucleic acid enzyme can comprise one or more “substrate binding domains” or “substrate binding arms” which comprise sequences that are substantially complementary to an oligonucleotide substrate and are capable of hybridizing to a substrate polynucleotide under conditions suitable for catalysis. The “enzymatic” or “catalytic” domain or sequence refers to the region or nucleotide sequence of a nucleic acid enzyme that catalyzes a chemical reaction and accordingly modifies an oligonucleotide that is hybridized to the substrate binding domain(s).

Examples of nucleic acid enzymes that can be used in the disclosed methods include but are not limited to naturally and non-naturally occurring nucleobase polymers comprising for example RNA, DNA, and nucleic acid enzymes comprising non-naturally occurring nucleotides, including D and/or L enantiomers and/or non-nucleotide moieties. Therefore, the skilled artisan will appreciate that nucleic acid enzymes include but are not limited to catalytic RNA, catalytic DNA, and mixed polymer enzymes (e.g., nucleobase polymers comprising ribonucleotides, deoxyribonucleotides and/or non-naturally occurring nucleotides (e.g., nucleozymes (see, e.g. U.S. Pat. Nos. 5,652,094, 6,140,491, 6,713,456; U.S. Application Nos. 20020102694, 20020137718, 20040072783; Srivatsan et al., 2003, J Inorg Biochem. 97(4):340-4; Zhang et al., 1998, Ann N Y Acad. Sci. 864:636-9)).

In some embodiments, a nucleic acid enzyme can be a ribozyme. “Ribozyme” and “RNA enzyme” (“Rz”) as used herein refer to a catalytic polynucleotide comprising a catalytic RNA sequence Therefore, the substrate binding arm(s) of a ribozyme can be any nucleobase polymer suitable for binding a substrate in a manner suitable for catalysis. Examples of catalytic RNA sequences include but are not limited to those found in hammerhead ribozymes (see, e.g., Haseloff et al., 1998, Nature 334:585-591; Rossi et al., 1992, Aids Research and Human Retroviruses 8:183; Intracellular Ribozyme Applications: Principles and Protocols Rossi and Courture (eds), 1999, (ISBN 1-898486-17-4)), hairpin ribozymes (see, e.g., EP0360257, Hampel et al., 1989, Biochemistry 28:4929; Hampel et al., 1990, Nucleic Acids Res. 18:299), hepatitis delta virus (HDV, see, e.g., Been, 1994, Trends Biochem/Sci. 19(6):251-6; Perrotta et al., 1992, Biochemistry 31:16), group I introns (see, e.g. U.S. Pat. No. 4,987,071; Landweber et al., 1998, BioScience 48:2; Zaug et al., 1986, Nature 324(6096):429-33 [Erratum in: Nature 1987 Feb. 12-18; 325(6105):646]), group II introns (see, e.g., Augustin et al., 1990, Nature 343(6256):383-6; Peebles et al., 1986, Cell 44(2):213-23), RNaseP RNA (see, e.g., Forster et al., 1990, Science 249:783; Guerrier-Takada et al., 1983, Cell 35:849), Neurospora VS RNA (Guo et al., 1995, EMBO J. 14:368; Guo et al., 2004, Mol. Cell. 16(3):351-62; Saville et al., 1990, Cell 61:685-696; Saville et al., 1991, Proc. Natl. Acad. Sci. USA 88:8826-8830), minizymes (see, e.g., U.S. Pat. Nos. 6,004,806, 6,083,744, 6277634, 6365730; Amontov et al., 1996, FEBS Lett. 386(2-3):99-102; Kuwabara et al., 1996, Nucleic Acids Res. 24(12):2302-10; Kuwabara et al., 1997, Nucleic Acids Symp Ser. (37):307-8), and leadzymes (see, e.g., Wedekind et al., 2003, Biochemistry 42(32):9554-63).

In some embodiments, a ribozyme can be half ribozymes (see, e.g., Kossen et al., 2004, Chem. Biol. 11:807-815; Rossi, 2004, Chem. Biol. 11:894-5). Half ribozymes comprise an enzymatic domain and non-enzymatic domain that are not covalently linked. Therefore, when half ribozyme sequences are employed in the disclosed methods the sequence tag can be added to either the enzymatic or non-enzymatic domain.

In some embodiments, a nucleic acid enzyme can be a DNA enzyme. “DNA enzyme”, DNAzyme”, and “deoxyribozyme” as used herein refer to a catalytic polynucleotide comprising a catalytic DNA sequence. Therefore, the substrate binding arm(s) of a DNA enzyme can be any nucleobase polymer suitable for binding a substrate in a manner suitable for catalysis. Non-limiting examples of DNA enzymes are disclosed in U.S. Application Nos. 20040072783, 20040191872; Breaker et al., 1994, Chem Biol. 1(4):223-9; Breaker, 1997, Nat. Biotechnol. 15(5):427-31; Breaker, 2000, Science 290(5499):2095-6; Burmeister et al., 1997, Angew. Chem., Int. Ed. Engl. 36:1321-1324; Cairns et al., 2000, Antisense Nucleic Acid Drug Dev. 10(5):323-32; Carmi et al., 1996, R. Chem. Biol. 3:1039-1046; Coppins et al., 2004, Nat. Struct. Mol. Biol. 11(3):270-4 (Epub 2004 Feb. 1); Cuenoud et al., 1995, Nature, 375:611-614; Emilsson et al., 2002, Cell Mol. Life Sci. 59(4):596-607; Feldman et al., 2001, J. Mol. Biol. 313(2):283-94; Flynn-Charlebois et al., 2003, J. Am. Chem. Soc. 125(18):5346-50; He et al., 2002, Biomacromolecules 3(1):69-83; Inui et al., 1999, Nucleic Acids Symp. Ser. (42):217-8; Jaschke, 2001, Curr. Opin. Struct. Biol. 11(3):321-6; Joyce, 2001, Methods Enzymol. 341:503-17; Levy et al., 2001, Bioorg. Med. Chem. 9:2581-2587; Li et al., 1996, Nat. Struct. Biol. 3:743-747; Li et al., 1999, Curr. Opin. Struct. Biol. 9(3):315-23; Li et al., 1999, Proc. Nat. Acad. Sci. USA 96:2746-2751; Li et al., 2000, Biochemistry 39:3106-3114; Li et al., 2000, Nucleic Acids Res. 28(2):481-8; Mei et al., 2003, J. Am. Chem. Soc. 125(2):412-20; Nowakowski et al., 1999, Acta Crystallogr. D. Biol. Crystallogr. 55(11):1885-92; Ordoukhanian et al., 1999, Chem. Biol. 6(12):881-9; Ordoukhanian et al., 2002, J. Am. Chem. Soc. 124(42):12499-506; Santoro et al., 1997, Proc. Natl. Acad. Sci. USA. 94(9):4262-6; Sen et al., Curr. Opin. Chem. Biol: 2(6):680-7; Santoro et al., 1998, Biochemistry 37(38):13330-42; Sheppard et al., 2000, Proc. Natl. Acad. Sci. USA. 97(14):7802-7; Silverman et al., 2004, Chem. Biol. 11(1):7-8; Sugimoto et al., 1999, Nucleic Acids Symp. Ser. (42):281-2; and Travascio et al., 1998, Chem. Biol. 5:505-517.

The nucleic acid enzyme used in the disclosed methods can be selected at the discretion of the practitioner. Generally, factors to be considered in selecting a nucleic acid enzyme include whether the nucleic acid enzyme sequence is catalytically active as DNA or RNA, the rate of the reaction with a specific substrate (k_{cat}), the background associated with the reaction, the presence or absence of potential inhibitors, the incubation time of the reaction, and/or the detection method. Determining the k_{cat} of enzymatic reactions with various substrates and inhibitors are within the abilities of the skilled artisan (Nelson and Cox. “Lehninger: Principles of Biochemistry” Third Edition pp. 243-292 (Worth Publishers, New York) (ISBN:1-57259-153-6). In various exemplary embodiments, the k_{cat} of a nucleic acid enzyme catalyzed reaction can be at least about 0.01, 0.05, 1, 5, 6, 7, 8, 9, 10, 20, 30, 40, 50, 60, 70, 80, 90, or 100 min⁻¹, or higher. In various exemplary embodiments, k_{cat} can be at least about 1 to at least about 10 min⁻¹ or greater than at least about 10 min⁻¹.

Selecting and optimizing the rate at which a nucleic acid enzyme catalyzes a reaction is within the abilities of the skilled artisan. Reaction parameters that can be used to modulate nucleic acid enzyme activity include but are not limited to temperature, ionic strength, pH, the concentration of co-factors (e.g., divalent cations) and the like. In some embodiments, the reaction rate can be modulated by modifying the length of the substrate binding arms of a nucleic acid enzyme. For example, a single-stranded extension of helix I or helix III of a hammerhead ribozyme by elongation of the substrate strand may cause a substantial and proportional decrease in the catalytic activity of a hammerhead ribozyme. In some embodiments, a decrease in cleavage rates of nucleic acid enzymes, such as, hammerhead ribozymes, also can be associated with long-chain substrates (Hormes et al., 2002, Biochimie 84:897-903).

In some embodiments, a nucleic acid enzyme can be modified by in vitro selection or evolution techniques to achieve optimum catalytic rates and/or specificity. In some embodiments, a nucleic acid enzyme having a desired activity can be isolated de novo from a library of randomized nucleic acid sequences and optionally can be optimized. In general, these techniques can involve multiple rounds of selection, amplification, and mutagenesis and have been used to generate synthetic nucleic acid enzymes that can catalyze an aminoacyl esterase reaction, primer extension, cleavage of a phosphorodithioate linkage, cleavage of a DNA substrate, calcium-dependent RNA cleavage, template directed RNA ligation, amide bond cleavage, aminoacylation, alkylation, sulfur alkylation, peptide-bond formation, and 2′,5′-phosphodiester cleavage. These methods also have produced nucleic acid enzymes having an altered regio- or enantioselectivity, that are capable of promoting synthesis of a purine nucleotides, or have at least one L-nucleotide substitution. Non-limiting examples of in vitro selection and evolution of nucleic acid enzymes are described in U.S. Pat. No. 6,251,666; Bartel et al., 1993, Science 261(5127):1411-8; Beaudry et al., 1992, Science 257(5070):635-41; Dai et al., 1995, Science 267(5195):237-40 [Erratum in: Science 1996 Apr. 5; 272(5258):18-9]; Derrick et al., 2000, Biochemistry 39(16):4947-54; Ekland et al., 1995, Science 269(5222):364-70; Glasner et al., 2000, Biochemistry 39(50):15556-62; Illangasekare et al., 1995, Science 267(5198):643-7; Joyece, 2004, Ann. Rev. Biochem. 73:791-836; Landweber et al., 1998, BioScience 48:2; Lau et al., 2004, J. Am. Chem. Soc. 126(48):15686-93; Lehman et al., 1993, Nature 361(6408):182-5; Lorsch et al., 1994, Nature 371(6492):31-6; McGinness et al., 2002, Chem. Biol. 9(3):297-307; McGinness et al., 2003, Chem. Biol. 10:5-14; Ordoukhanian et al., 2002, J. Am. Chem. Soc. 124:12499-506; Piccirilli et al., 1992, Science 256(5062):1420-4; Shih et al., 1999, RNA. 5:1140-8; Wecker et al., 1996, RNA 2(10):982-94); and Zou et al., 2004, J. Biol. Chem. 279(31):32063-70.

Assays for detecting the catalytic activity of nucleic acid enzymes known in the art are suitable for detecting the tagged nucleic acid enzymes as disclosed herein. In general, these assays employ oligonucleotide substrates comprising one or more reporter molecules or labels that are capable of producing detectable signals. As the skilled artisan will appreciate, following modification of the substrate by the tagged nucleic acid enzymes, the reporter molecules can be used to detect or monitor product (e.g., modified substrate) accumulation. For example, in some embodiments, an oligonucleotide substrate can be modified or acted on by a nucleic acid enzyme. The oligonucleotide substrate can comprise a reporter system that produces a detectable signal when the oligonucleotide substrate is modified. In various exemplary embodiments, a detectable signal can be the origination of a detectable signal or the modification of an existing signal. In various exemplary embodiments, modification of an existing signal can be an increase or decrease in intensity of an existing signal or a qualitative modification in a signal (e.g., a shift a wavelength of a fluorescence signal).

“Reporter molecule”, “detectable moiety,” “detection moiety” and “label” has used herein refer to a moiety that is capable of producing a detectable or identifiable signal using known detection systems (e.g., spectroscopic, radioactive, enzymatic, chemical, photochemical, biochemical, immunochemical, chromatographic or electrophoretic systems). Non-limiting examples of labels include isotopic labels (e.g., radioactive or heavy isotopes), magnetic labels, spin labels, electric labels, thermal labels, colored labels (e.g., chromophores), luminescent labels (e.g., fluorescers, chemiluminescers), enzyme labels (e.g., horseradish peroxidase, alkaline phosphatase, luciferase, β-galactosidase) (Ichiki, et al., 1993, J. Immunol. 150(12):5408-5417; Nolan, et al., 1988, Proc. Natl. Acad. Sci. USA 85(8):2603-2607)), antibody labels, chemically modifiable labels, and mobility modifier labels. In addition, in some embodiments, such labels include components of ligand-binding partner pairs.

“Ligand,” “binding partner” and “anti-ligand” as used herein refer to molecules that specifically interact with each other. “Specifically interact” refers to binding that is substantially distinctive and restricted, and sufficient to be sustained under conditions that inhibit non-specific binding. Non-limiting examples of ligands and binding partners include but are not limited to antigen-antibody (including single-chain antibodies and antibody fragments (e.g. Fab, Fab′, F(ab′)₂, Fv)), hormone-receptor, neurotransmitter-receptor binding, polymerase-promoter, biotin or iminobiotin, which can be bound by binding partners avidin, streptavidin, CaptAvidin biotin-binding proteins, and NeutrAvidin biotin-binding protein (Molecular Probes, Eugene, Oreg.), digoxigenin-anti-digoxigenin binding, carbohydrates which can be bound by lectin, galectins, C-type lectins, selectins, annexins, cholesterol and cholesterol binding compounds digitonin, tomatin, filipin, and amphotericin B; or a molecule that donates or accepts a pair of electrons to form a coordinate covalent bond with the central metal atom of a coordination complex. In various exemplary embodiments, the dissociation constant of the ligand/anti-ligand complex is less than about 10⁻⁴-10⁻⁹ M¹, less than about 10⁻⁵-10⁻⁹ M⁻¹ or less than about 10⁻⁷-10⁻⁹ M⁻¹.

“Fluorescent label,” “fluorescent moiety,” “fluorescer” and “fluorophore” as used herein refer to a molecule that may be detected via its inherent fluorescent properties. Examples of suitable fluorescent labels include, but are not limited to, fluorescein, rhodamine, tetramethylrhodamine, eosin, erythrosin, coumarin, methyl-coumarins, pyrene, Malachite Green, stilbene, Lucifer Yellow, Cascade BlueJ, Texas Red, IAEDANS, EDANS, BODIPY FL, LC Red 640, phycoerythrin, LC Red 705, Oregon green, Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE), FITC, Rhodamine, Texas Red (Pierce, Rockford, Ill.), Cy5, Cy5.5, Cy7 (Amersham Life Science, Pittsburgh, Pa.) and tandem conjugates, such as but not limited to, Cy5PE, Cy5.5PE, Cy7PE, Cy5.5APC, Cy7APC. In some embodiments, suitable fluorescent labels also include, but are not limited to, green fluorescent protein (GFP; Chalfie, et al., 1994, Science 263(5148):802-805), EGFP (Clontech Laboratories, Inc., Palo Alto, Calif.), blue fluorescent protein (BFP; Quantum Biotechnologies, Inc. Montreal, Canada; Heim et al., 1996, Curr. Biol. 6:178-182; Stauber, 1998, Biotechniques 24(3):462-471), enhanced yellow fluorescent protein (EYFP; Clontech Laboratories, Inc., Palo Alto, Calif.), and renilla (WO 9215673; WO 9507463; WO 9814605; WO 9826277; WO 9949019; U.S. Pat. Nos. 5,292,658, 5,418,155, 5,683,888, 5,741,668, 5,777,079, 5,804,387, 5,874,304, 5,876,995 and No. 5,925,558). Further examples of fluorescent labels are found in Haugland, Handbook of Fluorescent Probes and Research, 9th Edition, Molecule Probes, Inc. Eugene, Oreg. (ISBN 0 9710636-0 5).

In other embodiments, a fluorescent moiety may be an acceptor or donor molecule of a reporter system, including but not limited to, fluorescence energy transfer (FET) or fluorescent resonance energy transfer (FRET) systems, which utilize distance-dependent interactions between the excited states of two molecules in which excitation energy is transferred from a donor molecule to an acceptor molecule (see, e.g. U.S. Pat. Nos. 6,140,055, 6,201,113, 6,365,724, 6,451,535; U.S. Application No. 20030008315; Bustin., 2000, J. Mol. Endocrinol. 25:169-193; Cuenod et al., 1995, Nature 375:611-614; Fedor et al., 1990, Proc. Natl. Acad. Sci. USA 87:1668-1672; Forster, 1948, Annals of Physics (Leipzig) 2:55-75; Hendry et al., Characterizing ribozyme cleavage reactions, Turner (edt.) Humana Press: Totowa, N.J. (1997), vol. 74, 221-229; Jenne et al., 1999, Angew. Chem. Int. Edn. 38:1300-1303; Lee et al., 1993, Nucleic Acids Res. 21:3761-6; Livak et al., 1995, PCR Meth. Appin. 4:357-362; Lakowicz, Principles of Fluorescent Spectroscopy; Plenum Press, New York (1983); Mei et al., 2003, J. Am. Chem. Soc. 125:412-420; Todd et al., 2000, Clinical Chemistry 46(5):625;630; WO9429481; WO9945156; WO2004003510) As known in the art, these systems are suitable for detecting or monitoring changes in molecular proximity. Therefore, in embodiments, wherein an oligonucleotide substrate is cleaved by a tagged nucleic acid enzyme, a reporter system can be used to detect and/or monitor the reaction in real-time. Therefore, in some embodiments, a oligonucleotide substrate can comprise donor and acceptor moieties that in energy transfer proximity and flank the site of endonuclease cleavage by a tagged nucleic acid enzyme. Depending on the type of donor-acceptor moieties utilized, the release of the flap sequence may be detected or monitored by an increase or decrease in fluorescence signal. Examples of donor-acceptor pairs suitable for producing a fluorescent signal include but are not limited to fluorescein-tetramethylrhodamine, IAEDANS-fluorescein, EDANS-dabcyl, fluorescein-QSY 7, and fluorescein-QSY 9. Examples of donor-acceptor pairs suitable for quenching a fluorescent signal include but are not limited to FAM-TAMRA, FAM-DABCYL, HEX-DABCYL, TET-DABCYL, Cy3-DABCYL, Cy5-DABCYL, Cy5.5-DABCYL, rhodamine-DABCYL, fluorescein-DABCYL, 2-carboxyfluorescein-DABCYL, TAMRA-DABCYL, JOE-DABCYL, ROX-DABCYL, Cascade Blue-DABCYL, Bodipy-DABCYL, FAM-MGB, Vic-MGB, Ned-MGB, ROX-MGB, FAM-TAMRA. (see, e.g. Lakowicz, Principles of Fluorescence Spectroscopy, Plenum Publishing Corporation, NY (ISBN 0306460939).

In some embodiments, the production of a detectable signal can be linked to a reaction catalyzed by a tagged nucleic acid enzyme utilizing a NTP comprising a phosphatase activatable moiety at the γ-PO₄. In reactions that yield the β- and γ-phosphates of an NTP as pyrophosphate (e.g., polymerizations, reactions catalyzed by class I ligases, the label can be activated by reacting the pyrophosphate with phosphatase.

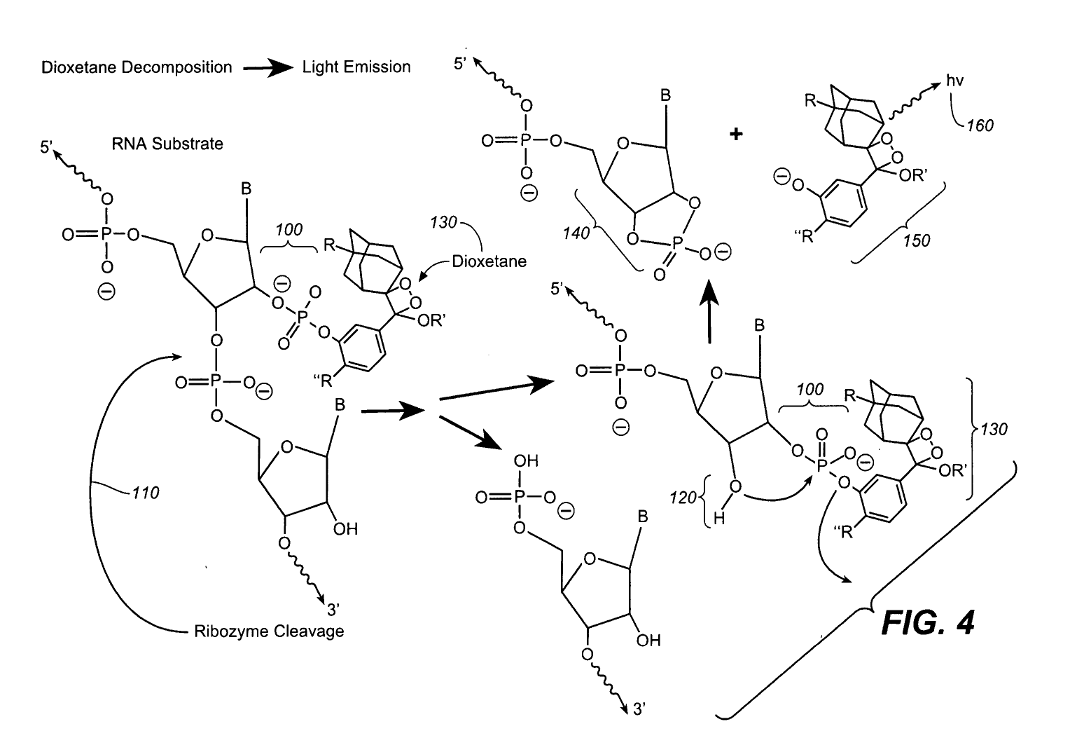

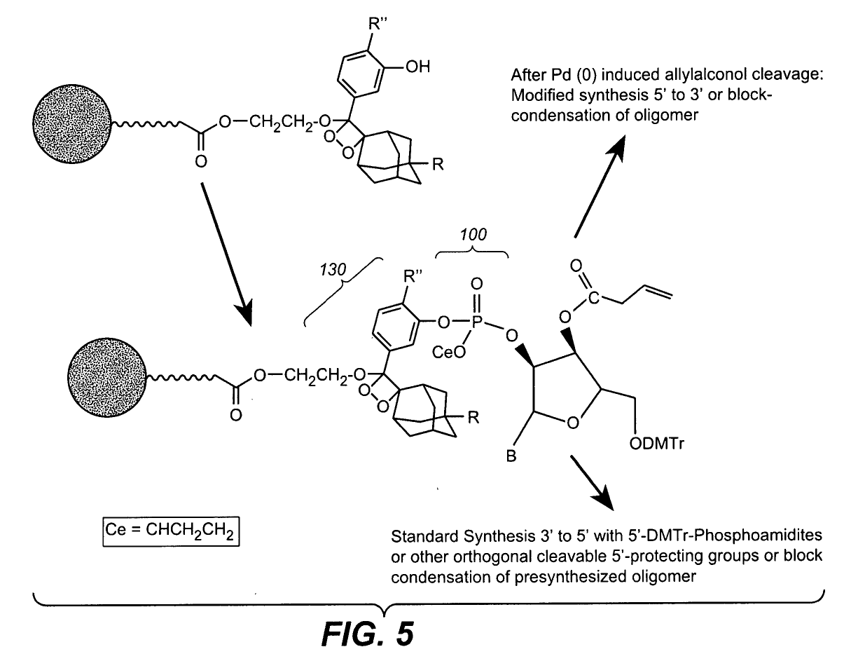

In some embodiments, catalysis by a tagged nucleic acid enzyme can be linked with the production of a chemiluminescent signal. For example, in some embodiments, an oligonucleotide substrate can comprise a moiety capable of producing a chemiluminescent signal when released from the oligonucleotide substrate but is not substantially detectable when attached to the substrate. In some embodiments, such a chemiluminescent moiety can comprise a dioxetane compound (see, e.g., U.S. Pat. Nos. RE36,536, 5,849,495, 5,851,771, 5,853,974, 5,856,522, 5,866,045, 5,866,389, 5,869,698, 5,869,699, 5,869,705, 5,871,938, 5,891,626, 5,892,064, 5,936,101, 5,936,132, 5,981,768, 5,994,073, 6,001,561, 6,001,659, 6,022,964, 6,036,892, 6,063,574, 6,107,024, 6,107,036, 6,113,816, 6,124,478, 6,133,459, 6,139,781, 6,140,495, 6,180,833, 6,218,135, 6,228,653, 6,243,980, 6,245,928, 6,284,899, 6,287,767, 6,322,727, 6,346,615, 6,353,129, 6,355,441, 6,410,751, 6,417,380, 6,451,531, 6,514,717, 6,555,698, 6,613,578, 6,660,529, 6,686,171, 6,747,160, 6,767,716). In some embodiments, an oligonucleotide substrate comprising a dioxetane can be synthesized according to the example shown in FIG. 5, in which a dioxetane can be attached via phosphate bridge 100 to the 2′ position of the nucleotide that is 5′ relative to the site of endonuclease cleavage. (see, e.g., Lyttle et al., 1996, Nucleic Acids Res. 24(14):2793-2798) When the labeled oligonucleotide substrate is cleaved 110 (FIG. 4), the resultant 3′-OH 120 attacks the adjacent 2′-phosphate bridge 100 to which the dioxetane 130 is attached. This intramolecular reaction yields a 2′,3′-cyclic phosphate 140 and a free dioxetane 150 which can emit light 160 (FIG. 4) and therefore can be detected using instrumentation known in the art.

In some embodiments, enzymatic activity can be assayed while the tagged nucleic acid is hybridized to a capture probe. “Capture probe” as used herein refers to a polynucleotide suitable for hybridizing to a tagged nucleic acid enzyme but is not a suitable substrate for the enzyme and does not substantially interfere with the catalytic activity of the enzyme. In some embodiments, capture probes can be complementary to the sequence tag and can be used to isolate a tagged nucleic acid enzyme. In some embodiments, capture probes can be non-diffusably bound to an insoluble support or surface. In some embodiments, a plurality of capture probes can be bound to an insoluble support in an array format. Arrays can be suitable for carrying out a large number of assays simultaneously using small amounts of reagents (see, e.g. Genome Survey Microarray, Applied Biosystems, Foster City, Calif.). Therefore, in some embodiments, capture probe arrays can be used in the multiplex analysis of tagged nucleic acid enzymes.

Insoluble supports may be made of any composition to which the capture probe can be bound and is otherwise compatible with assay conditions. The surface of such supports may be solid or porous and of any convenient shape. Examples of suitable insoluble supports include microtiter plates, membranes, beads, wafer, or chips. These typically can be made of glass, plastic (e.g., polystyrene), polysaccharides, nylon, nitrocellulose, teflon, flow through reactor system etc. (see, e.g., U.S. Pat. Nos. 5,405,783, 5,445,934, 5,510,270, 5,547,839, 6,232,062, 6,221,583, 6,309,822, 6,344,316, 6,355,431, 6,355,432, 6,368,799, 6,396,995, 6,410,229, 6,440,667, 6,576,425, 6,576,424, 6,600,031, 6,632,605, 6,646,243, 6,495,323, 6,667,394, 6,670,122, 6,686,150) The particular manner of binding the capture probe to a support is not crucial so long as it is compatible with the reagents and conditions of the enzymatic assay. For example, in some embodiments, the binding can include chemical crosslinking, direct binding to “sticky” or ionic supports, and/or the use of ligand/binding partners described above.

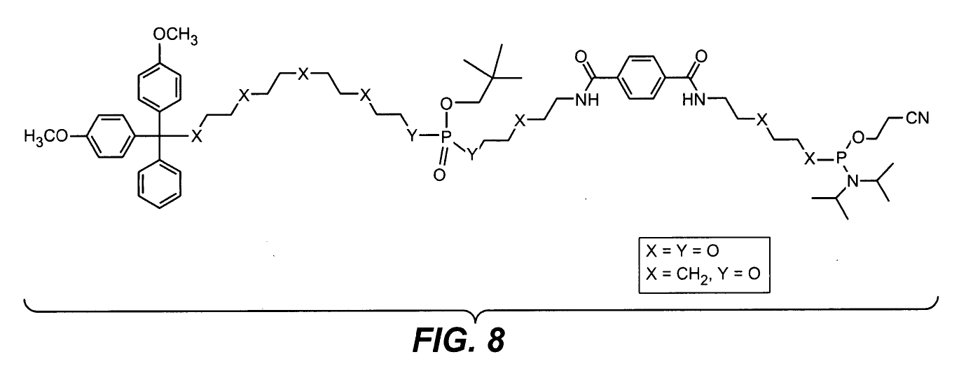

When assaying the enzymatic activities of a plurality of tagged nucleic acid enzymes hybridized to capture probes in an array format, the reaction products of each tagged nucleic acid enzyme including a detectable signal can be stabilized at each locus of the array. In some embodiments, this can be accomplished using a hydrophobic or lipophilic moiety that selectively precipitates the reaction products (i.e., a modified oligonucleotide substrate) of a tagged nucleic acid enzyme catalyzed reaction. For example, in some embodiments, an oligonucleotide substrate can comprise a hydrophobic moiety that does not substantially reduce the solubility of the substrate. When the substrate is cleaved by a tagged nucleic acid enzyme having endonuclease activity, the hydrophobic moiety can precipitate the cleavage product into the surface of the solid support. In various exemplary embodiments, a suitable hydrophobic moiety can be a polyethylene glycol, polymethylene, polystyrene, cholesterol, steroids, fatty acids, pyrene, psoralen, mobility modifiers (U.S. Pat. Nos. 5,470,705, 5,514,543, 6,395,486, and 6,734,296), PNAs, and phosphotriester (FIG. 8). Examples of suitable lipophilic moieties can be found at Glenres Research Catalog 2004 (Glenres Research, Sterling, Va.); Svinarchuk et al., 1993, Biochimie, 75(1-2):49-54, Misiura et al., 1998, Acta. Biochim. Pol. 45(1):27-32; Vives et al., 1999, Nucleic Acis, Res. 27(20):4071-4076; Crooke et al., 1996, Annu. Rev. Pharmacol. Toxicol. 36:107-129; Keller et al., 1993, Nucleic Acids Res. 21(19):4499-4505. In some embodiments, the precipitated cleavage product can comprise a reporter molecule (e.g., a fluorophore) which can be used to facilitate detection.

In some embodiments, reporter systems can be used that form an insoluble precipitate. In these systems, catalysis of a substrate results produces a product that forms an insoluble precipitate. In various exemplary embodiments such systems include but are not limited to NBT/BCIP which can be catalyzed by alkaline phosphatase (see, e.g., Cat No. 1 681 451, Roche Diagnostics GmbH, Mannheim, Germany) and TSA which can be catalyzed by HRP (see, e.g., Catalog No. MPS545, MICROMAX™ PC5269-0104, PerkinElmer Life and Analytical Sciences, Inc., Shelton, Conn.).

In some embodiments, the products of a reaction catalyzed by the disclosed tagged nucleic acid enzymes can be detected directly without the use of reporter molecules or reporter systems. In some embodiments, these methods can exploit the light absorptive properties of nucleic acids while separating the various nucleic acid (e.g., enzymes, substrates, and products) based on their size or molecular weight. Such methods include but are not limited to chromatography (e.g., HPLC, FPLC) and electrophoretic techniques (e.g., capillary electrophoresis (see, e.g., U.S. Pat. Nos. RE37,941, 6,372,106, 6,372,484, 6,387,234, 6,387,236, 6,402,918, 6,402,919, 6,432,651, 6,462,816, 6,475,361, 6,476,118, 6,485,626, 6,531,041, 6,544,396, 6,576,105, 6,592,733, 6,596,140, 6,613,212, 6,635,164, 6,706,162).

As will be appreciated by skilled artisans, target polynucleotides may comprise one or more target sequences and may be either DNA (e.g., cDNA, genomic DNA or extrachromosomal DNA) or RNA (e.g., mRNA, rRNA or genomic RNA) in nature, and may be derived or obtained from virtually any sample or source, wherein the sample may optionally be scarce or of a limited quantity. For example, the sample may be one or a few cells or a small amount of tissue collected via biopsy. In other embodiments, the target polynucleotide may be a synthetic polynucleotide comprising nucleotide analogs or mimics, as described below, produced for purposes, such as, diagnosis, testing, or treatment.

In various non-limiting examples, the target polynucleotide may be single or double-stranded or a combination thereof, linear or circular, a chromosome or a gene or a portion or fragment thereof, a regulatory polynucleotide, a restriction fragment from, for example, a plasmid or chromosomal DNA, genomic DNA, mitochondrial DNA, DNA from a construct or a library of constructs (e.g., from a YAC, BAC or PAC library), RNA (e.g., mRNA, rRNA or VRNA) or a cDNA or a cDNA library. As known in the art, a cDNA is a single- or double-stranded DNA produced by reverse transcription of an RNA template. Therefore, some embodiments include a reverse transcriptase (“RT”) and one or more “RT” primers suitable for reverse transcribing an RNA template into a cDNA. Reactions, reagents and conditions for carrying out such “RT” reactions are known in the art (see, e.g., Blain et al., 1993, J. Biol. Chem. 5:23585-23592; Blain et al., 1995, J. Virol. 69:4440-4452; PCR Essential Techniques 61-63, 80-81, (Burke, ed., J. Wiley & Sons 1996); Gübler et al., 1983, Gene 25:263-269; Gübler, 1987, Methods Enzymol., 152:330-335; Okayama et al., 1982, Mol. Cell. Biol. 2:161-170; Sellner et al., 1994, J. Virol. Method. 49:47-58; and U.S. Pat. Nos. 5,310,652, 5,322,770, and 6,300,073, these disclosures of which are incorporated herein by reference).

In some embodiments, a sample can comprise a single target polynucleotide from which one or more different target sequences of interest may be analyzed. In some embodiments, a sample can comprise a plurality of different target polynucleotides from which one or more different target sequences of interest may be analyzed. As will be recognized by skilled artisans, a sample may also include one or more polynucleotides comprising sequences that are not analyzed by the disclosed methods.

In some embodiments, highly complex mixtures of target sequences from highly complex mixtures of polynucleotides can be analyzed in either a singleplex or multiplex format. Indeed, many embodiments are suitable for multiplex analysis of target polynucleotides sequences from tens, hundreds, thousands, hundreds of thousands or even millions of polynucleotides. In some embodiments, the pluralities of target sequences from samples comprising cDNA libraries or total mRNA (e.g., the transcriptome) isolated or derived from biological samples, such as tissues and/or cells, wherein the cDNA or, alternatively, mRNA libraries may be quite large. For example, cDNA libraries or mRNA libraries constructed from several organisms or from several different types of tissues or organs can be detected according to the methods described herein.

As the skilled artisan will appreciate, in multiplex embodiments multiple sets of primers and/or probes and/or oligonucleotide substrates can be utilized for each target polynucleotide sequence to be detected. For example, in multiplex embodiments utilizing a reporter molecule, each reporter molecule can produce a signal that can be distinguishable (e.g., spectrally resolvable) from other reporter molecules. Therefore, in these embodiments, the number of target polynucleotides detected in a multiplex format can be determined, at least in part, by the number and type of reporter molecules that may be discriminated.

The amount of target polynucleotide(s) utilized in the disclosed methods can vary widely. For example, the target polynucleotide(s) may be from a single cell, from tens of cells, from hundreds of cells or even more. For many embodiments, including embodiments in which the target polynucleotide is a complex cDNA library (or derived therefrom by RT of mRNA), the total amount of target polynucleotide utilized may range from about 1 pg to about 100 ng. For some embodiments, including embodiments in which the target polynucleotide(s) is obtained from a single cell, the total amount of target polynucleotide(s) may range from 1 copy (about 10 ag) to about 10⁷ copies (about 100 pg). In some embodiments target polynucleotides may range from about 100 to about 10⁶ copies. The skilled artisan will appreciate that in various embodiments a greater number of target polynucleotides may be used or the number of target polynucleotides can be unknown.

In some embodiments, preparation of the target polynucleotide(s) for detection may not be required. In some embodiments, the target polynucleotide(s) may be prepared using conventional sample preparation techniques. For example, target polynucleotides may be isolated from their source (e.g., a biological sample) via chromatography, precipitation, electrophoresis, as is well-known in the art. Alternatively, the target sequence(s) may be detected directly from samples, including but not limited to, cells or from lysates of tissues or cells comprising the target polynucleotide(s). In some embodiments, one or more sequences of a target polynucleotide can be amplified, for example, by PCR to produce one or more populations of amplicons. Therefore, as used herein, a “target polynucleotide” may also refers to an amplified target sequence. Furthermore, in some embodiments, a target sequence may be amplified but multiple sets of primers. Examples of suitable amplification methods are well known in the art (see, e.g., U.S. Pat. Nos. 4,683,195, 4,683,202, 4,800,159, 4,965,188, 5,075,216, 5,176,995, 5,185,243, 5,386,022, 5,427,930, 5,516,663, 5,656,493, 5,679,524, 5,686,272, 5,869,252, 6,040,166, 6,197,563, 6,514,736, and EP-A-0200362, EP-A-0201184 and EP-A-320308).

Determining the sequence composition of the various domains of the various polynucleotides (e.g., primers (e.g., amplification primers, RT primers, complementary strand primers etc.), probes (ligation probes, capture probes), and sequence tags) suitable for use in the disclosed methods is within the abilities of the skilled artisan. Generally, each of these polynucleotides should be sufficiently to anneal to its complementary sequence under the reaction conditions in which they are employed. Therefore, exact lengths and compositions may depend on many factors, including but not limited to, the desired hybridization temperature, the complexity of the different target polynucleotide to be detected, the salt concentration, ionic strength, pH and other buffer conditions. The ability to select lengths and sequences of primers, probes, and tags suitable for particular applications is within the capabilities of ordinarily skilled artisans (see, e.g., Sambrook et al. Molecular Cloning: A Laboratory Manual 9.50-9.51, 11.46, 11.50 (2d. ed., Cold Spring Harbor Laboratory Press); Sambrook et al., Molecular Cloning: A Laboratory Manual 10.1-10.10 (3d. ed. Cold Spring Harbor Laboratory Press)). In some embodiments, sequences suitable for hybridization contain from about 15 to about 35 nucleotides that are suitable for hybridization and/or to form a substrate suitable for polymerization, amplification, and/or ligation, however in some embodiments more or fewer nucleotides can be used. The skilled artisan will appreciate that the sequence need only be of sufficient length and composition that is sufficient to provide hybridize under the conditions of the assay. Shorter primers generally require lower temperatures to form sufficiently stable hybrid complexes. The capability of polynucleotides to anneal can be determined by the melting temperature (“Tₘ”) of the hybrid complex. Tₘ is the temperature at which 50% of a polynucleotide strand and its perfect complement form a double-stranded polynucleotide. Therefore, the Tₘ for a selected polynucleotide varies with factors that influence or affect hybridization. In some embodiments, in which thermocycling occurs, the amplification primers should be designed to have a melting temperature (“Tₘ”) in the range of about 60-75° C. The actual temperature used annealing may depend upon, among other factors, the concentration of the various polynucleotides and complementary sequences (e.g., target polynucleotide, sequence tag) and the reaction conditions in which they are employed. Therefore, in various exemplary embodiments, a probe or primer can have a Tₘ in the range of about 35 to about 45° C., from about 40 to about 50° C., from about 45 to about 55° C., from about 50 to about 60° C., from about 55 to about 65° C., from about 60 to about 70° C., from about 65 to about 75° C., or from about 70 to about 80° C.

The Tₘ of a polynucleotide or sequence domain can be determined empirically utilizing melting techniques that are well-known in the art (see, e.g., Sambrook et al. Molecular Cloning: A Laboratory Manual 11.55-11.57 (2d. ed., Cold Spring Harbor Laboratory Press)). Alternatively, the Tₘ can be calculated. Numerous references and aids for calculating Tₘs of primers are available in the art and include, by way of example and not limitation, Baldino et al. Methods Enzymology. 168:761-777; Bolton et al., 1962, Proc. Natl. Acad. Sci. USA 48:1390; Bresslauer et al., 1986, Proc. Natl. Acad. Sci. USA 83:8893-8897; Freier et al., 1986, Proc. Natl. Acad. Sci. USA 83:9373-9377; Kierzek et al., Biochemistry 25:7840-7846; Rychlik et al., 1990, Nucleic Acids Res. 18:6409-6412 (erratum, 1991, Nucleic Acids Res. 19:698); Rychlik. J. NIH Res. 6:78; Sambrook et al. Molecular Cloning: A Laboratory Manual 9.50-9.51, 11.46-11.49 (2d. ed., Cold Spring Harbor Laboratory Press); Sambrook et al., Molecular Cloning: A Laboratory Manual 10.1-10.10 (3d. ed. Cold Spring Harbor Laboratory Press)); Suggs et al., 1981, In Developmental Biology Using Purified Genes (Brown et al., eds.), pp. 683-693, Academic Press; Wetmur, 1991, Crit. Rev. Biochem. Mol. Biol. 26:227-259, which disclosures are incorporated by reference.

As the skilled artisan will appreciate, in general, the relative stability and therefore, the Tₘs, of RNA:RNA, RNA:DNA, and DNA:DNA hybrids having identical sequences for each strand may differ. In general, RNA:RNA hybrids are the most stable (highest relative Tₘ) and DNA:DNA hybrids are the least stable (lowest relative Tₘ). Accordingly, in some embodiments, another factor to consider, in addition to those described above, when designing any primer or probe is the structure of the primer and target polynucleotide. For example, in embodiments in which an RNA polynucleotide is reverse transcribed to produce a cDNA, the determination of the suitability of a DNA primer for the reverse transcription reaction should include the effect of the RNA polynucleotide on the Tₘ of the primer. Although the Tₘs of various hybrids may be determined empirically, as described above, examples of methods of calculating the Tₘ of various hybrids are found at Sambrook et al. Molecular Cloning: A Laboratory Manual 9.51 (2d. ed., Cold Spring Harbor Laboratory Press).

“Polynucleotide or oligonucleotide” refers to nucleobase polymers or oligomers in which the nucleobases are connected by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof. In some embodiments, a polynucleotide can comprise a non-naturally occurring nucleotide.

By “nucleobase” is meant naturally occurring and synthetic heterocyclic moieties commonly known to those who utilize nucleic acid or polynucleotide technology or utilize polyamide or peptide nucleic acid technology to generate polymers that can hybridize to polynucleotides in a sequence-specific manner. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobases include those nucleobases disclosed in FIGS. 2(A) and 2(B) of Buchardt et al. (U.S. Pat. No. 6,357,163 and WO 9220702 and WO 9220703), Gunda et al., 2004, Angew.Chem. Int. Ed. 43:6372-6377; Graham et al., 1998, J. Chem. Soc., Perkin Trans. 1 1131; Seitz, 2003, Humboldt-Spektrum 2-3:96-99; Hikishima et al., 2005, Angew. Chem. Int. Ed. 44:596-598; and Hill et al. 2001, Chem. Rev. 101:3893-4001.

Nucleobases can be linked to other moieties to form nucleosides, nucleotides, and nucleoside/tide analogs. As used herein, “nucleoside” refers to a compound consisting of a purine, deazapurine, or pyrimidine nucleoside base, e.g., adenine, guanine, cytosine, uracil, thymine, 7-deazaadenine, 7-deazaguanosine, that is linked to the anomeric carbon of a pentose sugar at the 1′ position, such as a ribose, 2′-deoxyribose, or a 2′,3′-di-deoxyribose. When the nucleoside base is purine or 7-deazapurine, the pentose is attached at the 9-position of the purine or deazapurine, and when the nucleoside base is pyrimidine, the pentose is attached at the 1-position of the pyrimidine (see, e.g., Kornberg and Baker, DNA Replication, 2nd Ed. (W.H. Freeman & Co. 1992)). The term “nucleotide” as used herein refers to a phosphate ester of a nucleoside, e.g., a mono-, a di-, or a triphosphate ester, wherein the most common site of esterification is the hydroxyl group attached to the C-5 position of the pentose. “Nucleotide 5″-triphosphate” refers to a nucleotide with a triphosphate ester group at the 5′ position. The term “nucleoside/tide” as used herein refers to a set of compounds including both nucleosides and/or nucleotides.

“Nucleobase polymer or oligomer” refers to two or more nucleobases connected by linkages that permit the resultant nucleobase polymer or oligomer to hybridize to a polynucleotide having a complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyamide or peptide nucleic acids. Nucleobase polymers or oligomers can vary in size from a few nucleobases, from 2 to 40 nucleobases, to several hundred nucleobases, to several thousand nucleobases, or more.

In some embodiments, a nucleobase polymer is an polynucleotide analog or an oligonucleotide analog. By “polynucleotide analog or oligonucleotide analog” is meant nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. Nos. 6,013,785 and 5,696,253 (see also, Dagani, 1995, Chem. & Eng. News 4-5:1153; Dempey et al., 1995, J. Am. Chem. Soc. 117:6140-6141). Such positively charged analogues in which the sugar is 2′-deoxyribose are referred to as “DNGs,” whereas those in which the sugar is ribose are referred to as “RNGs.” Specifically included within the definition of poly- and oligonucleotide analogs are locked nucleic acids (LNAs; see, e.g., Elayadi et al., 2002, Biochemistry 41:9973-9981; Koshkin et al., 1998, J. Am. Chem. Soc. 120:13252-3; Koshkin et al., 1998, Tetrahedron Letters, 39:4381-4384; Jumar et al., 1998, Bioorganic & Medicinal Chemistry Letters 8:2219-2222; Singh and Wengel, 1998, Chem. Commun., 12:1247-1248; WO 0056746; WO 0228875; and, WO 0148190.

In some embodiments, a nucleobase polymer is a polynucleotide mimic or oligonucleotide mimic. By “polynucleotide mimic or oligonucleotide mimic” is meant refers to a nucleobase polymer or oligomer in which one or more of the backbone sugar-phosphate linkages is replaced with a sugar-phosphate analog. Such mimics are capable of hybridizing to complementary polynucleotides or oligonucleotides, or polynucleotide or oligonucleotide analogs or to other polynucleotide or oligonucleotide mimics, and may include backbones comprising one or more of the following linkages: positively charged polyamide backbone with alkylamine side chains as described in U.S. Pat. Nos. 5,786,461, 5,766,855, 5,719,262, 5,539,082 and WO 9803542 (see also, Haaima et al., 1996, Angewandte Chemie Int'l Ed. in English 35:1939-1942; Lesnick et al., 1997, Nucleosid. Nucleotid. 16:1775-1779; D'Costa et al., 1999, Org. Lett. 1:1513-1516; Nielsen, 1999, Curr. Opin. Biotechnol. 10:71-75); uncharged polyamide backbones as described in WO 9220702 and U.S. Pat. No. 5,539,082; uncharged morpholino-phosphoramidate backbones as described in U.S. Pat. Nos. 5,698,685, 5,470,974, 5,378,841 and 5,185,144 (see also, Wages et al., 1997, BioTechniques 23:1116-1121); peptide-based nucleic acid mimic backbones (see, e.g., U.S. Pat. No. 5,698,685); carbamate backbones (see, e.g., Stirchak and Summerton, 1987, J. Org. Chem. 52:4202); amide backbones (see, e.g., Lebreton, 1994, Synlett. February, 1994:137); methylhydroxylamine backbones (see, e.g., Vasseur et al., 1992, J. Am. Chem. Soc. 114:4006); 3′-thioformacetal backbones (see, e.g., Jones et al., 1993, J. Org. Chem. 58:2983) and sulfamate backbones (see, e.g., U.S. Pat. No. 5,470,967). All of the preceding references are herein incorporated by reference.

“Peptide nucleic acid” or “PNA” refers to poly- or oligonucleotide mimics in which the nucleobases are connected by amino linkages (uncharged polyamide backbone) such as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112 and 6,403,763; all of which are incorporated herein by reference. The term “peptide nucleic acid” or “PNA” shall also apply to any oligomer or polymer comprising two or more subunits of those polynucleotide mimics described in the following publications: Lagriffoul et al., 1994, Bioorganic & Medicinal Chemistry Letters, 4:1081-1082; Petersen et al., 1996, Bioorganic & Medicinal Chemistry Letters, 6:793-796; Diderichsen et al., 1996, Tett. Lett. 37:475-478; Fujii et al., 1997, Bioorg. Med. Chem. Lett. 7:637-627; Jordan et al., 1997, Bioorg. Med. Chem. Lett. 7:687-690; Krotz et al., 1995, Tett. Lett. 36:6941-6944; Lagriffoule et al., 1994, Bioorg. Med. Chem. Lett. 4:1081-1082; Diederichsen, 1997, Bioorg. Med. Chem. 25 Letters, 7:1743-1746; Lowe et al., 1997, J. Chem. Soc. Perkin Trans. 1, 1:539-546; Lowe et al., 1997, J. Chem. Soc. Perkin Trans. 11:547-554; Lowe et al., 1997, 1. Chem. Soc. Perkin Trans. 1 1:555-560; Howarth et al., 1997, 1. Org. Chem. 62:5441-5450; Altmann et al., 1997, Bioorg. Med. Chem. Lett., 7:1119-1122; Diederichsen, 1998, Bioorg. Med. Chem. Lett., 8:165-168; Diederichsen et al., 1998, Angew. Chem. mt. Ed., 37:302-305; Cantin et al., 1997, Tett. Lett., 38:4211-4214; Ciapetti et al., 1997, Tetrahedron, 53:1167-1176; Lagriffoule et al., 1997, Chemistry—A European Journal 3(6):912-919; Kumar et al., 2001, Organic Letters 3(9):1269-1272; and the Peptide-Based Nucleic Acid Mimics (PENAMs) of Shah et al. as disclosed in WO 9604000.

Some examples of PNAs are those in which the nucleobases are attached to an N-(2-aminoethyl)-glycine backbone, i.e., a peptide-like, amide-linked unit (see, e.g., U.S. Pat. No. 5,719,262; Buchardt et al., 1992, WO 9220702; Nielsen et al., 1991, Science 254:1497-1500).

In some embodiments, a nucleobase polymer is a chimeric oligonucleotide. By “chimeric oligonucleotide” is meant a nucleobase polymer or oligomer comprising a plurality of different polynucleotides, polynucleotide analogs, polynucleotide mimics, including but not limited to D and L enantiomers. For example a chimeric oligo may comprise a sequence of DNA linked to a sequence of RNA. Other examples of chimeric oligonucleotides include a sequence of DNA linked to a sequence of PNA, and a sequence of RNA linked to a sequence of PNA.

In some embodiments, a polynucleotide comprises one or more non-nucleobase moieties. Non-limiting examples of non-nucleobase moieties include but are not limited to a ligand, as described above, a “blocking moiety” suitable for inhibiting polymerase extension of the 3′ terminus of a probe when it is hybridized to a target sequence, and moieties suitable for producing a detectable signal, as described above.

Although the above described exemplary embodiments employ nucleozyme moieties or nucleic acid enzyme sequences, other types of nucleic acids that catalyze various types of reactions as known in the art can be used. Therefore, in some embodiments, catalytic aptomer sequences can be used rather nucleic acid enzyme sequences. “Aptamer” as used herein refers to a synthetic oligonucleotide that can specifically bind to a particular target molecule, such as a proteins, peptides, organic compounds, inorganic compounds, and pharmaceuticals. For example, aptamers have been shown to release a caged fluorophore resulting in the detection of a fluorescent signal. Thus, aptamers can be selected in vitro using techniques known in the art and find use in the disclosed methods. (see, e.g., Breaker et al., 200, Nat. Biotechnol. 15:427-431; Sen et al., 1998, Curr. Opin. Chem. Biol. 2:680-7; Li et al., 1999, Curr. Opin. Struct. Biol. (9:3115-323; Jaschke, 2001, Curr. Opin. Struct. Biol. 11:321-326; Emilson et al. Cell. Mol. Life Sci. 2002 59:596-702, Ellington et al., 1990, Nature 346:818-822; Tuerk et al., 1990, Science, 249:505-510; Famulok et al. 2001, Acc. Chem. Res. 33:591-599; WIlson et al. Ann. Rev. Biochem. 1999 68:611-647)

Therefore, in some embodiments, kits include a reaction vessel comprising one or more primers or probes, described above, suitable for producing a tagged nucleic acid enzymes comprising RNA, DNA, and mixed polymers in the presence of one or more target polynucleotides. In some embodiments, kits can include one or more capture probes attached to a surface. In some embodiments, a plurality of capture probes, each comprising, a substantially unique sequence can be attached to a surface. In some embodiments, kits can include a reaction vessel comprising one or more oligonucleotide substrates that are suitable for modification by tagged nucleic acid enzymes. In some embodiments, a kit includes one or more reporter molecules that produce a detectable signal proportional to the number of single-stranded amplicons.

In some embodiments, the method of assaying for the enzymatic activity of tagged nucleic acid enzymes may be implemented on a general purpose or special purpose device, such as a device having a processor for executing computer program code instructions and a memory coupled to a processor for storing data and/or commands. It will be appreciated that the computing device may be a single computer or a plurality of networked computers and that the several procedures associated with implementing the methods and procedures described herein may be implemented on one or a plurality of computing devices. In some embodiments the disclosed procedures and methods are implemented on standard server-client network infrastructures with the inventive features added on top of such infrastructure or compatible therewith.

Methods and procedures described herein generally may be implemented in software, hardware, or combinations thereof. Thus, in some embodiments, a device or apparatus comprises a temperature control system having a reaction module linked to thermal control module. A reaction module can be optically linked to an excitation source and detector. The type of excitation source and detection system are selected at the discretion of the practitioner and are based on the detection method, and number and type of signals produced by the reporter molecule(s) employed. The apparatuses are further adapted to be operably linked to a computer that is directed by readable memory. The output of the computer is directed to an output device. The skilled artisan will appreciate, that the various components of the apparatus may have other configurations. In some embodiments, excitation source and detector may be may be in separate housings. In some embodiments, a processor and output device can be in the same housing. Non-limiting examples of existing apparatuses that may be used to carry out detect and monitor the activities of tagged nucleic acid enzymes in real-time or take one or more single time point measurements include, Models 7300, 7500, and 7700 Real-Time PCR Systems (Applied Biosystems, Foster City, Calif.); the MyCyler and iCycler Thermal Cyclers (Bio-Rad, Hercules, Calif.); the Mx3000P™ and Mx4000® (Stratagene®, La Jolla, Calif.); the Chromo 4™ Four-Color Real-Time System, Opticon, Opticon2 (MJ Research, Inc., Reno, Nev.); and the LightCycler® 2.0 Instrument (Roche Applied Science, Indianapolis, Ind.).

All publications, patents, patent applications and other documents cited in this application are hereby incorporated by reference in their entireties for all purposes to the same extent as if each individual publication, patent, patent application or other document were individually indicated to be incorporated by reference for all purposes.

1. A method of detecting a target polynucleotide comprising:
a) contacting a sample with a forward primer under hybridization conditions, wherein said forward primer comprises in a 5′ to 3′ direction an RNA promoter sequence, a ribonucleozyme moiety, and a primer sequence that is complementary to a first sequence of a target polynucleotide in said sample,
b) enzymatically extending said forward primer to generate a first strand,
c) hybridizing said first strand to a reverse primer comprising a second sequence of said target polynucleotide,
d) extending said reverse primer to generate a double stranded polynucleotide,
e) transcribing said double stranded polynucleotide to yield a single stranded RNA comprising said ribonucleozyme moiety, and
f) detecting a chemiluminescent signal produced by a reporter system when an oligonucleotide substrate comprising said reporter system is acted on by said ribonucleozyme moiety, whereby said target polynucleotide is detected.

2. The method according to claim 1, wherein said reporter system comprises dioxetane.

3. The method according to claim 1, wherein said ribonucleozyme moiety comprises a hammerhead ribozyme sequence.

4. The method according to claim 1, wherein said oligonucleotide substrate comprises a deoxyribonucleotide.

5. The method according to claim 1, wherein said oligonucleotide substrate comprises a ribonucleotide.

6. The method according to claim 1, wherein said oligonucleotide substrate comprises a non-naturally occurring nucleotide.

7. The method according to claim 1, wherein said oligonucleotide substrate further comprises a moiety that forms a precipitate when said ribonucleozyme moiety acts on said oligonucleotide substrate, and wherein said chemiluminescent signal emanates from said precipitate.

8. The method according to claim 1, wherein said ribonucleozyme moiety acts to cleave said oligonucleotide substrate.

9. The method according to claim 1, further comprising hybridizing said RNA to a capture probe.

10. A method of detecting a target polynucleotide comprising:
a) contacting a sample with a forward primer under hybridization conditions, wherein said forward primer comprises in a 5′ to 3′ direction an RNA promoter sequence, a ribonucleozyme moiety, and a primer sequence that is complementary to a first sequence of a target polynucleotide in said sample,
b) enzymatically extending said forward primer to generate a first strand,
c) hybridizing said first strand to a reverse primer comprising a second sequence of said target polynucleotide,
d) extending said reverse primer to generate a double stranded polynucleotide,
e) transcribing said double stranded polynucleotide to yield a single stranded RNA comprising said ribonucleozyme moiety, and
f) hybridizing said RNA to a capture probe under conditions suitable for detecting a signal produced by a reporter system when an oligonucleotide substrate comprising said reporter system is acted on by said ribonucleozyme moiety, whereby said target sequence is detected.

11. The method according to claim 10, wherein said signal is a fluorescent signal.

12. The method according to claim 10, wherein said signal is a chemiluminescent signal.

13. The method according to claim 10, wherein said oligonucleotide substrate further comprises a moiety that forms a precipitate when said ribonucleozyme moiety acts on said oligonucleotide substrate, and wherein said signal emanates from said precipitate.

14. The method according to claim 10, wherein said reporter system comprises a fluorescer-quencher pair.

15. The method according to claim 10, wherein said reporter system comprises dioxetane.

16. The method according to claim 10, wherein said ribonucleozyme moiety comprises a hammerhead ribozyme sequence.

17. The method according to claim 10, wherein said oligonucleotide substrate comprises a deoxyribonucleotide.

18. The method according to claim 10, wherein said oligonucleotide substrate comprises a ribonucleotide.

19. The method according to claim 10, wherein said oligonucleotide substrate comprises non-naturally occurring nucleotide.

20. The method according to claim 10, wherein said ribonucleozyme moiety acts to cleave said oligonucleotide substrate.

21. A method of detecting a target polynucleotide comprising:
a) contacting a sample comprising a target polynucleotide with first and second ligation probes, wherein said first probe comprises a 5′-phosphate, and in 5′ to 3′ direction a sequence complementary to said target polynucleotide and a sequence complementary to an RNA promoter sequence, and wherein said second probe comprises a 3′-hydroxyl and in 3′ to 5′ direction a second sequence complementary to said target polynucleotide and a sequence complementary to a ribonucleozyme moiety, wherein said first probe hybridizes to said target sequence 3′ relative to said second probe under conditions suitable for said probes to be ligated to form a transcription template,
b) hybridizing an oligonucleotide comprising an RNA promoter sequence to said template to form a double-stranded RNA promoter,
c) transcribing said template to yield a single stranded RNA comprising said ribonucleozyme moiety, and
d) detecting a signal produced by a reporter system when an oligonucleotide substrate comprising said reporter system is acted on by said ribonucleozyme moiety, whereby said target polynucleotide is detected.

22-97. (canceled)
